(19) United States
(12) Patent Application Publication
(10) Pub. No.: US 2017/0102398 A1
(43) Pub. Date: Apr. 13, 2017

(54) PREDICTING AND REDUCING COGNITIVE DECLINE BASED ON GSK-3 LEVELS

(71) Applicant: HUMANETICS CORPORATION, Edina, MN (US)

(72) Inventors: Michael D. KAYTOR, Maplewood, MN (US); John C. DYKSTRA, Minneapolis, MN (US)

(21) Appl. No.: 15/122,991

(22) PCT Filed: Mar. 5, 2015
(86) PCT No.: PCT/US15/18929

§ 371 Date: Sep. 1, 2016

Related U.S. Application Data
(60) Provisional application No. 61/948,328, filed on Mar. 5, 2014.

(51) Int. Cl. G01N 33/68; A23L 33/105; A61K 31/075

(52) U.S. Cl. CPC G01N 33/6896; A61K 31/075; A23L 33/105; G01N 2333/912; A23V 2002/00

(57) ABSTRACT

Materials and methods for predicting cognitive decline based on total or active/inactive levels of markers such as GSK-3 are provided. Materials and methods for preventing or delaying the onset of cognitive decline in individuals identified as being in need thereof also are provided.

### CROSS-REFERENCE TO RELATED APPLICATIONS

This application claims benefit of priority from U.S. Provisional Application Ser. No. 61/948,328, filed Mar. 5, 2014.

### TECHNICAL FIELD

This document relates to materials and methods for predicting cognitive decline based on GSK-3 levels, and to materials and methods for preventing or delaying the onset of cognitive decline.

### BACKGROUND

Excess body weight, a high fat diet, a sedentary lifestyle, genetic predisposition, and normal aging can contribute to the development of insulin resistance and subsequent type 2 diabetes, which in turn can lead to amyloidosis-related neurodegenerative disorders [e.g., mild cognitive impairment (MCI) or Alzheimer's disease (AD)]. To date, no pharmacotherapeutic has proven to be useful for reversing or curing such neurodegenerative disorders, and all Phase III trials have failed. This indicates a need for early intervention, likely decades before the onset of neurodegeneration. Despite the known contributing factors, including those listed above, it is difficult to determine who is at risk of developing these disorders.

### SUMMARY

This document is based in part on the identification of glycogen synthase kinase-3 (GSK-3) and related proteins as markers for risk of age-related cognitive decline. The materials and methods described herein can be used to prevent or delay onset of disorders such as dementia, MCI, and AD, thus providing an avenue for intervention much earlier than standard treatment after onset of neurodegenerative symptoms.

In one aspect, this document features a method for promoting neurological health in a subject. The method can include (a) obtaining a measured level of GSK-3 in a biological sample from the subject, wherein the measured level of GSK-3 is the level of total GSK-3, the level of phosphorylated GSK-3, the level of non-phosphorylated GSK-3, the ratio of phosphorylated GSK-3:total GSK-3, or the ratio of phosphorylated GSK-3:non-phosphorylated GSK-3, (b) comparing the measured level of GSK-3 to a corresponding control level of GSK-3, and (c) if the measured level is at least 25 percent higher or lower than the control measured level, treating the subject with D-pinitol.

In some embodiments, the measured level can be the ratio of phosphorylated GSK-3:total GSK-3, and the method can include treating the subject with D-pinitol when the ratio of phosphorylated GSK-3:total GSK-3 is at least 25 percent lower than the control measured level. In some embodiments, the measured level can be the total level of GSK-3, and the method can include treating the subject with D-pinitol when the total level of GSK-3 is at least 25 percent higher than the control measured level. The biological sample can contain platelets or peripheral blood lymphocytes. The D-pinitol can be contained in a composition that further includes a pharmaceutically acceptable carrier, in a dietary supplement, in a medical food, or in a food for special dietary use. The method can include treating the subject with an amount of D-pinitol that is effective to alter the measured level of GSK-3 in a second biological sample from the subject to a level that is not more than ten percent higher or lower than the control measured level. The subject can be diagnosed as being overweight or pre-diabetic, or as having MCI or AD.

In another aspect, this document features the use of a composition containing D-pinitol for promoting neurological health in a subject identified as having a biological sample in which a measured level of GSK-3 is at least 25 percent higher or lower than a control measured level GSK-3, wherein the measured level of GSK-3 is the level of total GSK-3, the level of phosphorylated GSK-3, the level of non-phosphorylated GSK-3, the ratio of phosphorylated GSK-3:total GSK-3, or the ratio of phosphorylated GSK-3:non-phosphorylated GSK-3.

The measured level can be the ratio of phosphorylated GSK-3:total GSK-3, and the subject can be identified as having a biological sample in which the ratio of phosphorylated GSK-3:total GSK-3 is at least 25 percent lower than the control measured level. The measured level can be the total level of GSK-3, and the subject can be identified as having a biological sample in which the total level of GSK-3 is at least 25 percent higher than the control measured level. The biological sample can contain platelets or peripheral blood lymphocytes. The composition can further include a pharmaceutically acceptable carrier. In some embodiments, the composition can be formulated as a dietary supplement, a medical food, or a food for special dietary use. The composition can contain an amount of D-pinitol that is effective to alter the measured level of GSK-3 in a second biological sample from the subject to a level that is not more than ten percent higher or lower than the control measured level. The subject can be identified as being overweight or pre-diabetic, or as having MCI or AD.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting age-related cognitive function for normal subjects (upper line) and for subjects at risk of age-related cognitive decline based on their weight and pre-diabetic or diabetic status (lower line). The time course for development of MCI and AD also is indicated.

FIG. 2 is a diagram showing that insulin resistance promotes amyloidogenesis and tangle formation, in part by inducing γ-secretase and tau phosphorylation through mechanisms that involve insulin receptor (IR) signaling.

FIG. 3 is a diagram depicting the mechanism by which D-pinitol acts as an insulin mimetic to re-sensitize insulin receptor signal transduction, thus inhibiting GSK-3, and reducing γ-secretase activity, formation of AP peptides, and neurofibrillary tangles.

### DETAILED DESCRIPTION

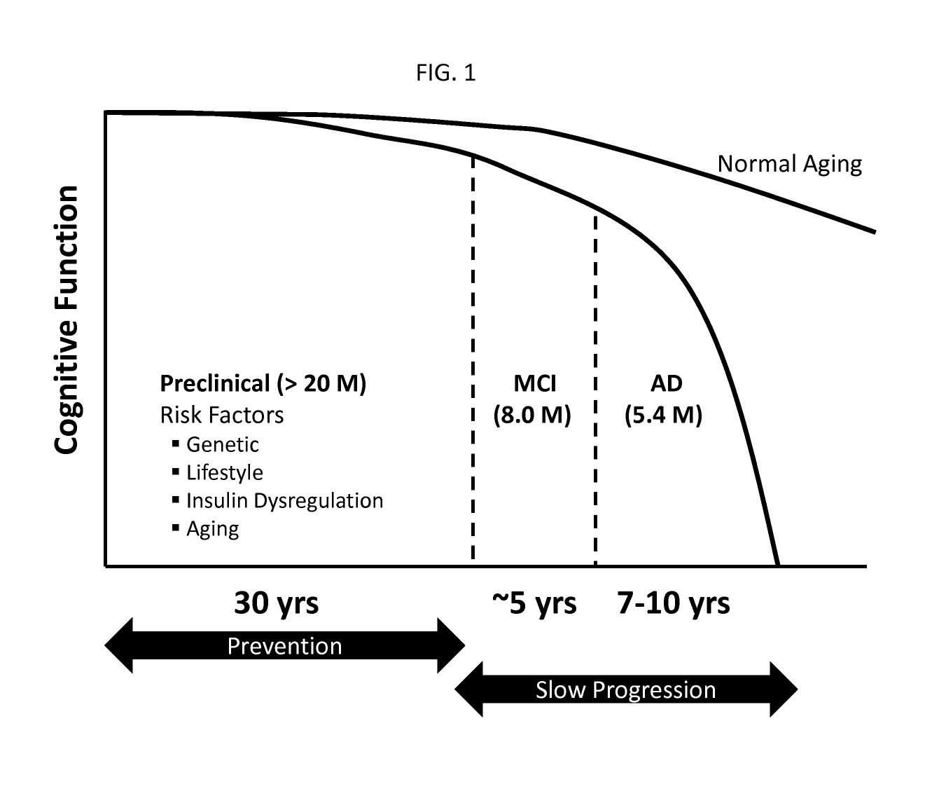

AD is the most prevalent form of dementia, and the sixth leading cause of death in the United States (2013 Alzheimer's Disease Facts and Figures, Alzheimer's Association, 2013). Diagnosis is followed by years of progressive decline in mental and functional abilities (FIG. 1), and AD thus represents a considerable burden on the nation's healthcare system. No cure for AD exists, and unless effective therapies and prevention strategies are developed, AD is projected to affect over 14 million Americans by 2050 (2013 Alzheimer's Disease Facts and Figures, supra).

Despite intense activity, no new drugs have been approved for AD in the past decade. The main focus of drug development efforts has been to target the structural hallmarks of AD-Aβ peptide-containing amyloid deposits and neurofibrillary tangles (NFTs) containing hyperphosphorylated forms of tau. These have met with limited success due to safety concerns and/or lack of efficacy.

D-pinitol (3-O-methyl-D-chiro-inositol; C₇H₁₄O₆) is a naturally-occurring compound that is found in dietary legumes and other plant-derived sources such as carob fruit, pine needles, chick peas, Bougainvillea leaves, and alfalfa. D-pinitol has insulin-like effects in vitro and in vivo, and plants that produce D-pinitol have been used in traditional Ayurvedic medicine for many years, particularly to treat diabetic symptoms. The use of D-pinitol for treating disorders associated with insulin resistance also is described in WO 96/29063, for example.

D-pinitol can improve glycemic control in diabetic rats and humans (Kim et al., Diabetes Res Clin Pract 77 Suppl 1:S247-251, 2007; Kim et al., Ann Nutr Metab 60:1-5, 2012; and Bates et al., Br J Pharmacol 130:1944-1948, 2000). In particular, D-pinitol can reduce serum glucose, hemoglobin Alc (HbAlc), and insulin in healthy and diabetic subjects (Kim et al. (2007) supra; Kim et al. (2012) supra; Hernandez-Mijares et al., Food Chem 141:1267-1272, 2013; Kang et al., J Med Food 9:182-186, 2006; and Kim et al., Eur J Clin Nutr 59:456-458, 2005), although these effects are dependent on dosing (Davis et al., Diabetes Care 23:1000-1005, 2000; and Campbell et al., J Nutr 134:2998-3003, 2004). In the body, D-pinitol may be converted to D-chiro-inositol (DCI).

D-pinitol enters cells via a transporter that is highly specific for D-oriented inositols. Once internalized, D-pinitol acts as an insulin mimetic downstream of the insulin receptor. It may feed into the signaling pathway triggered by autophosphorylation of the insulin receptor (Pitt et al., FASEB J 27:199-207, 2013), although it may also mimic the effects of the natural second messenger DCI glycan, which engages a signaling pathway independent of insulin receptor tyrosine phosphorylation (Lamer et al., Mol Med 16:543-552, 2010). Whatever the mechanism, D-pinitol activates the phosphatidylinositol 3-kinase (PI3-K)-Akt signaling pathway, which has been shown to inactivate GSK-3 (Bates et al., supra; and Yoshizaki et al., J Biol Chem 279:22715-22726, 2004). D-pinitol also directly inhibits γ-secretase while sparing the notch transduction pathways.

Based on history of use, as well as animal and human clinical studies, D-pinitol is non-toxic and safe for humans. For example, D-pinitol is present at about 1% dry weight in soy (Davis et al., supra). In 1994, about 2 million metric tons of soy were consumed by a total population of about 200 million people in Indonesia (Davis et al., supra), which converts to an average intake of about 250-300 mg of D-pinitol per day (about 4 mg/kg/day for a 70 kg individual). Although crude soy preparations might not provide the most readily assimilated form of D-pinitol, it is clear that D-pinitol consumption through soy is substantial.

Additional studies of D-pinitol's safety and efficacy included mutagenicity (AMES) tests, which were negative. Further, in acute toxicity studies, the LD50 in rats was shown to be >2 g/kg. Pre-clinical research demonstrated the ability of D-pinitol to improve glucose transport, and several clinical studies have demonstrated D-pinitol to be safe for oral administration. For example, a study of subjects with insulin resistance, dosed daily with 20 mg/kg of D-pinitol for 28 days, revealed a 48-fold increase in plasma D-pinitol concentration as compared to baseline (Davis et al., supra). In a phase I safety and pharmacokinetic study of D-pinitol in AD subjects, the time to peak plasma levels was about 90 minutes after administration, and the elimination half-life (t_{1/2}) was 10 hours.

GSK-3 is a serine/threonine protein kinase that mediates the addition of phosphate molecules onto serine and threonine residues. In addition to being a regulatory kinase for glycogen synthase, GSK-3 has been identified as a kinase for dozens of different proteins in a variety of pathways (see, e.g., Jope and Johnson, Trends Biochem Sci 29 (2): 95-102, 2004). Protein phosphorylation by GSK-3 typically inhibits the activity of the phosphorylated protein (Woodgett, Semin Cancer Biol 5(4):269-275, 1994; Woodgett, Sci STKE 2001(100):RE12, 2001; and Ali et al., Chem Rev 101(8):2527-2540, 2001). GSK-3 is active in a number of central intracellular signaling pathways, including cellular proliferation, migration, inflammation and immune responses, glucose regulation, and apoptosis. In mammals, there are two GSK-3 isoforms, which are encoded by the GSK-3α (GSK-3A) and GSK-3β (GSK-3B) genes. Either or both isoforms can be measured in the methods described herein. GSK-3 has been implicated in a number of diseases, including type 2 diabetes, AD, inflammation, cancer, and bipolar disorder (see, e.g., Hye et al., Neurosci Lett 373:1-4, 2005; Armentero et al., Neurobiol Aging 32:2142-2151, 2011; and Platenik et al., Prog Neuro-Psychopharmacol Biol Psych 50:83-93, 2014).

Studies using an AD mouse model demonstrated that diet-induced insulin resistance in the brain resulted in altered regulation of GSK-3α and GSK-3β phosphorylation, possibly through decreased AKT/PKB activity that led to GSK-3 activation (see, U.S. Pat. No. 8,193,250). More specifically, it was suggested that insulin resistance may specifically promote GSK-3α and GSK-3β activity by attenuating AKT/PKB-mediated pS²¹-GSK-3α and pS⁹-GSK-3β phosphorylation. Other studies in this mouse model investigated the role of insulin resistance on γ-secretase activity in the brain and its relationship with GSK-3α and GSK-3β activities. Results from these studies supported the idea that GSK-3α and GSK-3β activation (reflected by decreased pS²¹-GSK-3α and pS⁹-GSK-3β phosphorylation) may be a mechanism through which insulin resistance promotes the generation of Aβ peptides in the brain.

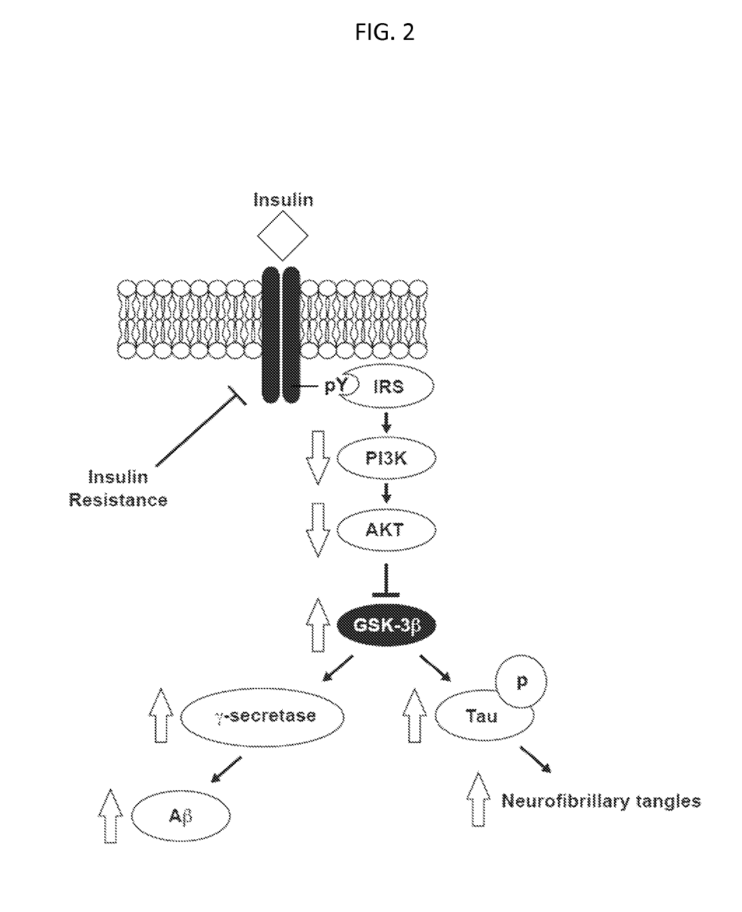

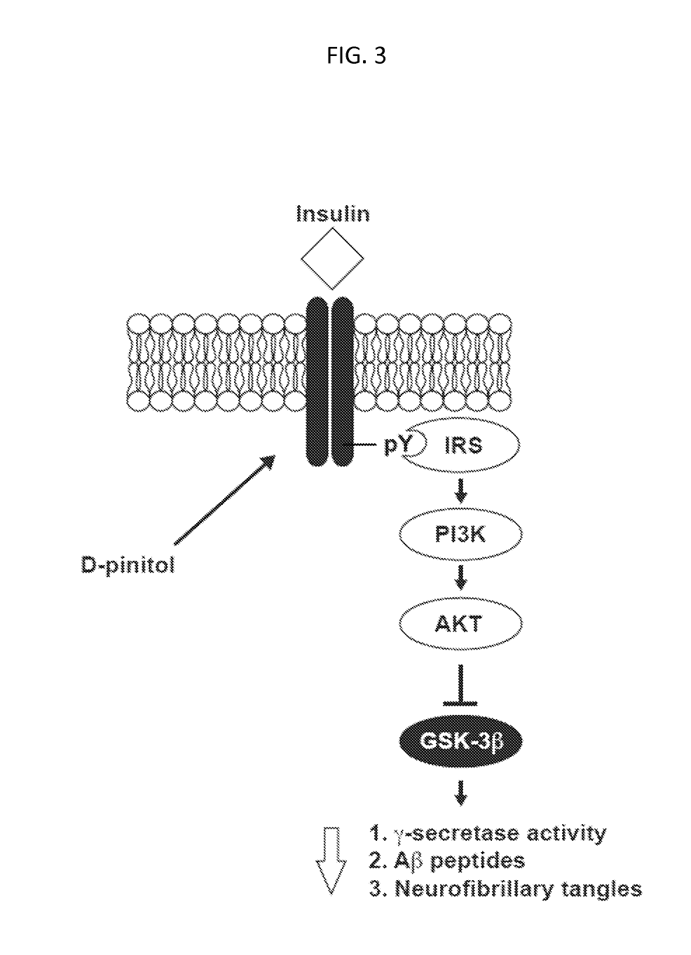

As depicted in FIG. 2, insulin resistance may promote amyloidogenesis and tangle formation at least in part by inducing γ-secretase and Tau phosphorylation via mechanisms that involve IR signaling. FIG. 3 shows a mechanism by which D-pinitol may act as an insulin mimetic to re-sensitize insulin receptor signal transduction, thus inhibiting GSK-3, and reducing γ-secretase activity, formation of Aβ peptides, and NFTs.

The methods provided herein can include identifying a subject as being at increased risk for cognitive decline (e.g., through being overweight, pre-diabetic, or diagnosed with MCI or AD), based on the level of a marker such as GSK-3 (e.g., total GSK-3, phosphorylated GSK-3, non-phosphorylated GSK-3, or the ratio of phosphorylated to non-phosphorylated or total GSK-3) in a biological sample obtained from the subject. Other markers, such as Akt, Tau, insulin receptor substrate (IRS-1), insulin receptor (IR), insulin-like growth factor 1 receptor (IGF-1R), PRAS40, or p70S6K, also may be useful predictors of cognitive decline in the methods provided herein. These other markers can be reported as total protein or, where applicable, total phosphorylated protein, total non-phosphorylated protein, or the ratio of phosphorylated to non-phosphorylated or total protein. The methods provided herein also can include treating a subject identified as being at increased risk of cognitive decline with D-pinitol or another insulin mimetic.

Thus, in some embodiments, this document provides methods for promoting neurological health, methods for preventing or delaying the onset of cognitive decline, and methods for reducing the rate of cognitive decline. The methods can include, for example, (a) obtaining a measured level of a marker (e.g., GSK-3, Akt, Tau, IRS-1, IR, IGF-1R, PRAS40, or p70S6K) in a biological sample from a subject (e.g., a human), (b) comparing the measured level of the marker to a corresponding control level of the marker (e.g., the level of the marker in normal subjects), and (c) treating the subject with D-pinitol if the measured level of the marker is higher or lower than a predetermined threshold (e.g., if the measured level is at least 10 percent, at least 20 percent, at least 25 percent, at least 30 percent, at least 40 percent, at least 50 percent, at least 60 percent, at least 70 percent, at least 80 percent, at least 90 percent, or at least 100 percent higher than a control measured level, or if the measured level is at least 10 percent, at least 20 percent, at least 25 percent, at least 30 percent, at least 40 percent, at least 50 percent, at least 60 percent, at least 70 percent, at least 80 percent, or at least 90 percent lower than a control measured level). The measured level of the marker can be from any suitable biological sample, although samples containing blood cells (e.g., peripheral blood lymphocytes or platelets) can be particularly useful. The measured level of the marker can be the total level of the marker (including both phosphorylated and non-phosphorylated molecules), the level of the non-phosphorylated marker, or the level of the phosphorylated marker. In some embodiments, the measured level can be the ratio of phosphorylated marker to non-phosphorylated marker or to total marker. The control level can correspond to the measured level (e.g., can be the total level of the marker, the level of the non-phosphorylated marker, the level of the phosphorylated marker, or the ratio of phosphorylated marker to non-phosphorylated marker or to total marker in), but can be obtained from normal subjects (e.g., subjects who do not have or are not thought to be at risk for cognitive decline).

In some embodiments, for example, a method can include obtaining a measured level of total or non-phosphorylated GSK-3 in a biological sample from a subject, comparing the measured level to a corresponding control level (e.g., the level of total or non-phosphorylated GSK-3 in normal individuals), and treating the subject with D-pinitol if the measured level is higher than a predetermined threshold (e.g., at least 25 percent higher than the control level, or at least 50 percent higher than the control level).

In some embodiments, a method can include obtaining a measured level of phosphorylated GSK-3, or the ratio of phosphorylated GSK-3 to total or non-phosphorylated GSK-3 in a biological sample from a subject, comparing the measured level to a corresponding control level (e.g., the level of phosphorylated GSK-3 or the ratio of phosphorylated GSK-3 to total or non-phosphorylated GSK-3 in normal individuals), and treating the subject with D-pinitol if the measured level is lower than a predetermined threshold (e.g., at least 25 percent lower than the control level, or at least 50 percent lower than the control level).

In some embodiments, a method can include obtaining a measured level of phosphorylated Tau, or the ratio of phosphorylated Tau to total or non-phosphorylated Tau in a biological sample from a subject, comparing the measured level to a corresponding control level (e.g., the level of phosphorylated Tau or the ratio of phosphorylated Tau to total or non-phosphorylated Tau in normal individuals), and treating the subject with D-pinitol if the measured level is higher than a predetermined threshold (e.g., at least 25 percent higher than the control level, or at least 50 percent higher than the control level).

In some embodiments, a method can include obtaining a measured level of phosphorylated Akt, or the ratio of phosphorylated Akt to total or non-phosphorylated Akt in a biological sample from a subject, comparing the measured level to a corresponding control level (e.g., the level of phosphorylated Akt or the ratio of phosphorylated Akt to total or non-phosphorylated Akt in normal individuals), and treating the subject with D-pinitol if the measured level is lower than a predetermined threshold (e.g., at least 25 percent lower than the control level, or at least 50 percent lower than the control level).

In some embodiments, a method can include obtaining a measured level of phosphorylated IRS-1, or the ratio of phosphorylated IRS-1 to total or non-phosphorylated IRS-1 in a biological sample from a subject, comparing the measured level to a corresponding control level (e.g., the level of phosphorylated IRS-1 or the ratio of phosphorylated IRS-1 to total or non-phosphorylated IRS-1 in normal individuals), and treating the subject with D-pinitol if the measured level is lower than a predetermined threshold (e.g., at least 25 percent lower than the control level, or at least 50 percent lower than the control level).

In some embodiments, a method can include obtaining a measured level of phosphorylated IR, or the ratio of phosphorylated IR to total or non-phosphorylated IR in a biological sample from a subject, comparing the measured level to a corresponding control level (e.g., the level of phosphorylated IR or the ratio of phosphorylated IR to total or non-phosphorylated IR in normal individuals), and treating the subject with D-pinitol if the measured level is lower than a predetermined threshold (e.g., at least 25 percent lower than the control level, or at least 50 percent lower than the control level).

In some embodiments, a method can include obtaining a measured level of IGF-1R in a biological sample from a subject, comparing the measured level to a corresponding control level (e.g., the level of IGF-1R in normal individuals), and treating the subject with D-pinitol if the measured level is higher than a predetermined threshold (e.g., at least 25 percent higher than the control level, or at least 50 percent higher than the control level).

In some embodiments, a method can include obtaining a measured level of phosphorylated PRAS40, or the ratio of phosphorylated PRAS40 to total or non-phosphorylated PRAS40 in a biological sample from a subject, comparing the measured level to a corresponding control level (e.g., the level of phosphorylated PRAS40 or the ratio of phosphorylated PRAS40 to total or non-phosphorylated PRAS40 in normal individuals), and treating the subject with D-pinitol if the measured level is higher than a predetermined threshold (e.g., at least 25 percent higher than the control level, or at least 50 percent higher than the control level).

In some embodiments, a method can include obtaining a measured level of phosphorylated p70S6K, or the ratio of phosphorylated p70S6K to total or non-phosphorylated p70S6K in a biological sample from a subject, comparing the measured level to a corresponding control level (e.g., the level of phosphorylated p70S6K or the ratio of phosphorylated p70S6K to total or non-phosphorylated p70S6K in normal individuals), and treating the subject with D-pinitol if the measured level is higher than a predetermined threshold (e.g., at least 25 percent higher than the control level, or at least 50 percent higher than the control level).

Methods for obtaining measured levels of polypeptide markers are well known in the art. For example, immunological methods (e.g., enzyme-linked immunosorbent assay (ELISA) and Western blotting) are well established methods for assessing the level of particular polypeptides, and antibodies specific for particular polypeptide antigens are commercially available. See, e.g., Table 1.

TABLE 1

| Antigen | Vendor |
|---|---|
| Akt | Cell Signaling Technology, Santa Cruz |
| | Biotechnology |
| pS⁴⁷³-Akt | Cell Signaling Technology |
| pT³⁰⁸-Akt | Santa Cruz Biotechnology |
| Tau | Santa Cruz Biotechnology |
| pS²⁰²-Tau | Invitrogen |
| pS³⁹⁶-Tau | Thermo Scientific |
| pS⁴⁰⁴-Tau | Thermo Scientific |
| pT²³¹-Tau | Calbiochem |
| GSK-3β | Cell Signaling Technology |
| pS²¹-GSK-3α | Sigma-Aldrich |
| pS⁹-GSK-3β | Cell Signaling Technology, BD Transduction |
| | Laboratories |
| IRS1 | Santa Cruz Biotechnology |
| p-IRS1 (various) | Cell Signaling Technology |
| IR | Santa Cruz Biotechnology |
| IGF-1R | Santa Cruz Biotechnology |
| PRAS40 | Cell Signaling Technology |
| pT²⁴⁶-PRAS40 | Cell Signaling Technology |
| p70S6K | Cell Signaling Technology |
| pT³⁸⁹-p70S6K | Cell Signaling Technology |

The D-pinitol can be administered to the subject in any suitable form and at any suitable dosage. A “therapeutically effective amount” is an amount of D-pinitol that is effective to delay or prevent the onset of cognitive decline, to slow the rate of cognitive decline, or to result in a measured level of a marker (e.g., GSK-3, including non-phosphorylated GSK-3, phosphorylated GSK-3, total GSK-3, or the ratio of phosphorylated GSK-3 to total or non-phosphorylated GSK-3) that is not more than 25 percent (e.g., not more than 20 percent, 15 percent, 10 percent, or 5 percent) higher or lower than a corresponding control level of the marker.

Thus, in some embodiments, when the marker is total or non-phosphorylated GSK-3, treatment with a therapeutically effective amount of D-pinitol can result in a measured level that is not more than 25 percent (e.g., not more than 10 percent, or not more than five percent) higher than the corresponding control level of total or non-phosphorylated GSK-3.

In some embodiments, when the marker is phosphorylated GSK-3, or the ratio of phosphorylated GSK-3 to total or non-phosphorylated GSK-3, treatment with a therapeutically effective amount of D-pinitol can result in a measured level that is not more than 25 percent (e.g., not more than 10 percent, or not more than five percent) lower than the corresponding control level of phosphorylated GSK-3 or ratio of phosphorylated GSK-3 to total or non-phosphorylated GSK-3.

In some embodiments, when the marker is phosphorylated Tau, or the ratio of phosphorylated Tau to total or non-phosphorylated Tau, treatment with a therapeutically effective amount of D-pinitol can result in a measured level that is not more than 25 percent (e.g., not more than 10 percent, or not more than five percent) higher than the corresponding control level of phosphorylated Tau or ratio of phosphorylated Tau to total or non-phosphorylated Tau.

In some embodiments, when the marker is phosphorylated Akt, or the ratio of phosphorylated Akt to total or non-phosphorylated Aid, treatment with a therapeutically effective amount of D-pinitol can result in a measured level that is not more than 25 percent (e.g., not more than 10 percent, or not more than five percent) lower than the corresponding control level of phosphorylated Akt or ratio of phosphorylated Akt to total or non-phosphorylated Md.

In some embodiments, when the marker is phosphorylated IRS-1, or the ratio of phosphorylated IRS-1 to total or non-phosphorylated IRS-1, treatment with a therapeutically effective amount of D-pinitol can result in a measured level that is not more than 25 percent (e.g., not more than 10 percent, or not more than five percent) lower than the corresponding control level of phosphorylated IRS-1 or ratio of phosphorylated IRS-1 to total or non-phosphorylated IRS-1.

In some embodiments, when the marker is phosphorylated IR, or the ratio of phosphorylated IR to total or non-phosphorylated IR, treatment with a therapeutically effective amount of D-pinitol can result in a measured level that is not more than 25 percent (e.g., not more than 10 percent, or not more than five percent) lower than the corresponding control level of phosphorylated IR or ratio of phosphorylated IR to total or non-phosphorylated IR in normal individuals.

In some embodiments, when the marker is IGF-1R, treatment with a therapeutically effective amount of D-pinitol can result in a measured level that is not more than 25 percent (e.g., not more than 10 percent, or not more than five percent) higher than the corresponding control level of IGF-1R.

In some embodiments, when the marker is phosphorylated PRAS40, or the ratio of phosphorylated PRAS40 to total or non-phosphorylated PRAS40, treatment with a therapeutically effective amount of D-pinitol can result in a measured level that is not more than 25 percent (e.g., not more than 10 percent, or not more than five percent) higher than the corresponding control level of phosphorylated PRAS40 or ratio of phosphorylated PRAS40 to total or non-phosphorylated PRAS40.

In some embodiments, when the marker is phosphorylated p70S6K, or the ratio of phosphorylated p70S6K to total or non-phosphorylated p70S6K, treatment with a therapeutically effective amount of D-pinitol can result in a measured level that is not more than 25 percent (e.g., not more than 10 percent, or not more than five percent) higher than the corresponding control level of phosphorylated p70S6K or ratio of phosphorylated p70S6K to total or non-phosphorylated p70S6K.

The dose of D-pinitol can vary, depending on the measured level of the marker, the frequency of administration, and the manner of administration, for example. In some embodiments, D-pinitol can be administered in an initial larger dose, followed by smaller maintenance doses. The D-pinitol can be administered on a weekly, biweekly, or monthly basis, or can be administered more often (e.g., semi-weekly, daily, or more than once a day, such as twice or three times daily). The duration of administration can range from months to years (e.g., three to six months, six to 12 months, more than one year, more than three years, or more than five years), and in some cases, the duration of administration can last for the remainder of a subject's lifetime.

The route of administration can depend on whether local or systemic treatment is desired, and on the area to be treated. For example, D-pinitol can be formulated for oral administration or parenteral administration (e.g., by subcutaneous, intrathecal, intraventricular, intramuscular, or intraperitoneal injection, or by intravenous drip), or by a combination of routes such as oral and parenteral administration. Administration can be rapid (e.g., by injection) or can occur over a period of time (e.g., by slow infusion or administration of slow release formulations, such as from subcutaneous drug depots, slow short term intravenous injections, or slow release oral formulations).

In some embodiments, D-pinitol can be included in a pharmaceutical composition that further includes a pharmaceutically acceptable carrier. Pharmaceutically acceptable carriers, excipients, and diluents suitable for therapeutic use include those described, for example, in Remington's Pharmaceutical Sciences, Maack Publishing Co. (A. R. Gennaro (ed.), 1985). For example, pharmaceutically acceptable carriers can include sterile water, aqueous dextrose and glycerol solutions, physiologically buffered saline, Hank's solution, and Ringer's solution, and/or buffers such as citrate buffers, phosphate buffers, tris(hydroxymethyl) aminomethane (TRIS) buffers, and/or borate buffers, to achieve a desired pH and osmolality. Injectable pharmaceutical formulations typically have a pH in the range of about 2 to about 12.

Compositions and formulations for parenteral administration include, for example, sterile solutions (e.g., sterile aqueous solutions or suspensions) that also can contain buffers, diluents, and/or other suitable additives (e.g., penetration enhancers, carrier compounds and other pharmaceutically acceptable carriers). Compositions formulated for parenteral delivery can be manufactured according to standard methods to provide sterile compositions deliverable via, for example, intravenous injection or infusion, intravascular injection, subcutaneous injection, or intramuscular injection. A D-pinitol formulation can be prepared to have a viscosity suitable for the desired route of parenteral administration, and can be manufactured and packaged in any manner suited to the desired application, including, without limitation, as a formulation deliverable via intravenous injection or infusion, intravascular injection, subcutaneous injection, or intramuscular injection. In some embodiments, a formulation as described herein can be contained in one or more pre-filled syringes or auto-injectors prepared for administration of a given dose or range of doses of D-pinitol.

Compositions also can be formulated for oral administration. Compositions and formulations for oral administration include, for example, powders or granules, suspensions or solutions in water or non-aqueous media (e.g., suspensions of D-pinitol nanoparticles in edible oil), capsules, sachets, and tablets. In some embodiments, a D-pinitol composition can be prepared as a liquid suspension that can be metered to deliver a desired dose, or can be incorporated into capsules (e.g., gelatin or soft capsules) suitable for delivery of liquid formulations. Alternatively, formulations for oral administration can be loaded into prefilled sachets or premetered dosing cups. In some embodiments, such formulations also can include one or more pharmaceutically acceptable sweetening agents, preservatives, dyestuffs, flavorings, or any combination thereof.

Compositions useful in the methods described herein can further include any pharmaceutically acceptable D-pinitol salts, esters, or salts of such esters, or any other D-pinitol compound that, upon administration to an animal such as a human, is capable of providing (directly or indirectly) biologically active D-pinitol or an active metabolite or residue thereof. Accordingly, for example, provided herein are pharmaceutically acceptable salts of D-pinitol, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of D-pinitol (e.g., salts that retain the desired biological activity of D-pinitol without imparting undesired toxicological effects). Examples of pharmaceutically acceptable salts may include, for example, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), and salts formed with organic acids (e.g., glucuronic acid, acetic acid, citric acid, oxalic acid, palmitic acid, or fumaric acid).

In some embodiments, a composition can contain D-pinitol at an amount between about 50 mg and about 10 g (e.g., about 100 mg, about 250 mg, about 500 mg, about 750 mg, about 1 g, about 2 g, about 3 g, about 4 g, about 5 g, about 6 g, about 7 g, about 8 g, about 9 g, about 50 mg to about 250 mg, about 100 mg to about 750 mg, about 200 mg to about 1 g, about 500 mg to about 2.5 g, or about 1 g to about 10 g).

In some embodiments, the D-pinitol can be a component of a dietary supplement or a medical food (e.g., in combination with an edible oil), or a food for special dietary use. Medical foods are, by definition, formulated to be consumed or administered enterally under the supervision of a physician or other medical professional. The manufacture of medical foods typically is done in compliance with FDA Good Manufacturing Practices (GMP) requirements. The term “food for special dietary use” is defined by 21 C.F.R. §105.3, as applied to food for humans, as meaning
particular (as distinguished from general) uses of food, as follows:
(i) Uses for supplying particular dietary needs which exist by reason of a physical, physiological, pathological or other condition, including but not limited to the conditions of diseases, convalescence, pregnancy, lactation, allergic hypersensitivity to food, underweight, and overweight;
(ii) Uses for supplying particular dietary needs which exist by reason of age, including but not limited to the ages of infancy and childhood;
(iii) Uses for supplementing or fortifying the ordinary or usual diet with any vitamin, mineral, or other dietary property. Any such particular use of a food is a special dietary use, regardless of whether such food also purports to be or is represented for general use.
   Methods for making dietary supplements, medical foods, and foods for special dietary use are known in the art. D-pinitol is soluble in water, and can be extracted from, for example, carob.

In some embodiments, the methods provided herein can include treating the subject with an amount of D-pinitol that is effective to alter (e.g., increase or reduce) the measured level of the marker in the subject to a predetermined acceptable level (e.g., a measured level that is not more than 5 percent, not more than 10 percent, not more than 15 percent, not more than 20 percent, or not more than 25 percent higher or lower than a control measured level), as described above, for example. Thus, in some cases the methods provided herein can further include (d) obtaining a second measured level of the marker in a second biological sample from the subject, where the second biological sample was obtained after the treating step, and (e) comparing the second measured level to the control measured level and/or to the first measured level to determine whether D-pinitol treatment has been effective. In addition, if the second measured level is unchanged with respect to the first measured level, or if it is still not close enough to the predetermined acceptable level (e.g., not more than 5 percent, not more than 10 percent, not more than 15 percent, not more than 20 percent, or not more than 25 percent higher or lower than the control measured level), the methods provided herein also can include adjusting the D-pinitol dosage or frequency of administration. For example, the dosage can be increased (e.g., 2-fold, 3-fold, 5-fold, 10-fold, or more than 10-fold) as compared to the dosage used for previous treatments), or the D-pinitol can be administered more frequently).

In some embodiments, if the second measured level is sufficiently altered as compared to the first measured level (e.g., if the second measured level is within about five or ten percent of the control measured level), the methods provided herein also can include adjusting the D-pinitol dosage or frequency of administration. For example, the dosage can be decreased (e.g., by 25 percent or 50 percent) as compared to the dosage used for previous treatments, or the D-pinitol can be administered less frequently.

This document also provides for the use of the compositions disclosed herein for promoting neurological health, or in the manufacture of a medicament for promoting neurological health. For example, a composition containing D-pinitol as disclosed herein can be used to promote neurological health in an individual identified as having a biological sample in which a measured level of a marker (e.g., GSK-3, Tau, Aid, IRS-1, IR, IGF-1R, PRAS40, or p70S6K) is higher or lower than a predetermined threshold (e.g., if the measured level is at least 10 percent, at least 20 percent, at least 25 percent, at least 30 percent, at least 40 percent, at least 50 percent, at least 60 percent, at least 70 percent, at least 80 percent, at least 90 percent, or at least 100 percent higher than a control measured level, or at least 10 percent, at least 20 percent, at least 25 percent, at least 30 percent, at least 40 percent, at least 50 percent, at least 60 percent, at least 70 percent, at least 80 percent, or at least 90 percent, lower than a control measured level).

The subject can be, for example, a person identified as being at risk for cognitive decline, such as a subject who is overweight or pre-diabetic, or who has MCI or AD. As for the methods described above, the measured level of the marker can be the level of total marker or, where applicable, the level of phosphorylated marker, the level of non-phosphorylated marker, the ratio of phosphorylated marker:total marker, or the ratio of phosphorylated marker:non-phosphorylated marker. In some embodiments, the composition can contain an amount of D-pinitol that is effective to alter the measured level of the marker in a biological sample from the individual to a predetermined acceptable level (e.g., a measured level that is not more than 5 percent, not more than 10 percent, not more than 15 percent, not more than 20 percent, or not more than 25 percent higher or lower than the control measured level).

The invention will be further described in the following examples, which do not limit the scope of the invention described in the claims.

### EXAMPLES

### Example 1—Assessing Effects of D-Pinitol on Kinase Activity in Platelets In Vitro

Experiments are conducted to identify readily accessible candidate biomarkers of the effects of D-pinitol on GSK-3-related processes. Secondary objectives include annotating biomarkers for their potential to predict progression from overweight and/or pre-diabetic to type 2 diabetic, type 2 diabetic to MCI, or MCI to AD, which may be of value in patient selection for longitudinal studies, and for informing trial design (e.g., with regard to dosing, sampling, and duration).

Freshly isolated platelets from normal donors are prepared and incubated with varying concentrations of D-pinitol (e.g., 0.1 μM, 10 μM, 50 μM, 100 μM, or 200 μM in water or PBS) or control (a GSK-3 inhibitor such as CHIR 90021 at 0.1 μM, 0.5 μM, 1 μM, 5 μM, or 10 μM; available from Tocris Bioscience, Bristol, UK) for various lengths of time (e.g., 0, 1, 5, 10, 30, 60, or 90 minutes). Experiments are conducted in parallel. Cells are lysed following treatment, and changes in the levels of selected (phospho)proteins (e.g., Akt, pS⁴⁷³-Akt, Tau, pS^{396/404}-Tau, pT²³¹-Tau, GSK-3β, pS⁹GSK-3β-, IRS-1, and pY-IRS-1) are assessed by Western blot analysis in parallel or in sequence (by stripping and reprobing the membrane). Representative antibodies that may be useful for in platelet protein immunoblotting are listed in Table 1. All experiments are repeated at least in triplicate, ideally with three different platelet donors. All experiments include vehicle control(s). Comparator compound concentrations are optimized using pS⁹-GSK-3 as a readout.

Levels of phosphorylated and total GSK-3 protein (“phospho/protein”) are quantitated (e.g., using beta-actin as a loading control), followed by densitometric scanning. Calculations are carried out to determine absolute levels of phospho/protein, relative levels of phospho/protein compared to baseline, and relevant ratios of phosphoprotein:protein (e.g., pS⁹-GSK:total GSK) for GSK-3, Tau, IRS-1, and Akt are calculated.

### Example 2—Evaluating the Effects of D-Pinitol on Peripheral Biomarkers of Age-Related Cognitive Decline In Vivo

A clinical study is conducted to quantify the effect of chronic (12 week) administration of a D-pinitol on expression level, relative to baseline, of peripheral biomarkers of the insulin signaling pathway and related signal transduction pathways. As set forth below, the study is conducted to in a wide-range of study participants (e.g., normal subjects, and subjects at risk for or having cognitive decline, including subjects diagnosed with type 2 diabetes, MCI, and/or mild to moderate AD). An exemplary set of cohorts is:

Cohort 1: Healthy volunteers

Cohort 2: Confirmed type 2 diabetics (e.g., fasting blood glucose>126 mg/dL)

Cohort 3: Clinically diagnosed MCI patients

Cohort 4: Mild to moderate AD (e.g., having a Mini Mental State Examination (MMSE) score of 10-26)

The study is designed as a three-month open-label study with 20 evaluable subjects per cohort. Study participants consume D-pinitol twice a day (morning and evening, approximately 12 hours apart) for the duration of the trial. Each dose contains 2.5 g of D-pinitol. A baseline blood draw is collected (plasma and buffy coat), and subsequent blood draws are collected every two weeks for the duration of the study. The outline of the blood draws is shown below.

Day 1: Blood draw (baseline), fasting glucose (baseline), HbAlc (baseline)

Day 14: Blood draw, D-pinitol trough level

Day 28: Blood draw, fasting glucose, HbA1c

Day 42: Blood draw

Day 56: Blood draw, fasting glucose, HbA1c

Day 70: Blood draw

Day 84: Blood draw, fasting glucose, HbA1c, D-pinitol trough level

Plasma is analyzed for D-pinitol levels. A trough level is determined at the beginning and at the end of the study. Fasting blood glucose and HbA1c are measured at baseline and monthly. Protein lysates from the buffy coat (WBCs and platelets) collected at the various time points are analyzed using Luminex assays for total signaling proteins and for the phosphorylated forms. Luminex multiplex and uniplex immunoassays are used to quantify the levels of signal transduction proteins. A list of signaling proteins that are quantified in the protein lysates are shown below.

1) Insulin receptor (IR)

2) Insulin-like growth factor 1 receptor (IGF-1R)

3) Insulin receptor substrate 1 (IRS-1)

4) Protein kinase B (Akt)

5) Proline-rich Akt substrate of 40 kDa (PRAS40)

6) p70S6 Kinase (p70S6K)

7) Glycogen synthase kinase 3β (GSK-3β)

8) Tau protein

The levels of signal transduction proteins are normalized to a loading control such as β-actin. The ratio of phosphorylated signaling protein to total protein is determined for each marker, for each subject, at each time point. These outcome measures are used to determine the extent that D-pinitol modulates expression of the peripheral markers in the different groups of study participants. The expression profile of peripheral biomarkers may identify at-risk individuals and those with early AD, when the disease is likely to be more responsive to treatment. Such biomarkers also may serve as surrogate endpoints in clinical studies of AD therapies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for promoting neurological health in a subject, comprising:
obtaining a measured level of glycogen synthase kinase-3 (GSK-3) in a biological sample from the subject, wherein the measured level of GSK-3 is the level of total GSK-3, the level of phosphorylated GSK-3, the level of non-phosphorylated GSK-3, the ratio of phosphorylated GSK-3:total GSK-3, or the ratio of phosphorylated GSK-3:non-phosphorylated GSK-3;
comparing the measured level of GSK-3 to a corresponding control level of GSK-3; and
if the measured level is at least 25 percent higher or lower than the control measured level, treating the subject with D-pinitol.

2. The method of claim 1, wherein the measured level is the ratio of phosphorylated GSK-3:total GSK-3, and wherein the method comprises treating the subject with D-pinitol when the ratio of phosphorylated GSK-3:total GSK-3 is at least 25 percent lower than the control measured level.

3. The method of claim 1, wherein the measured level is the total level of GSK-3, and wherein the method comprises treating the subject with D-pinitol when the total level of GSK-3 is at least 25 percent higher than the control measured level.

4. The method of claim 1, wherein the biological sample comprises platelets or peripheral blood lymphocytes.

5. The method of claim 1, wherein the D-pinitol is contained in a composition that further comprises a pharmaceutically acceptable carrier.

6. The method of claim 1, wherein the D-pinitol is contained in a dietary supplement.

7. The method of claim 1, wherein the D-pinitol is contained in a medical food.

8. The method of claim 1, wherein the D-pinitol is contained in a food for special dietary use.

9. The method of claim 1, comprising treating the individual with an amount of D-pinitol that is effective to alter the measured level of GSK-3 in a second biological sample from the subject to a level that is not more than ten percent higher or lower than the control measured level.

10. The method of claim 1, wherein the subject is diagnosed as being overweight or pre-diabetic, or as having mild cognitive impairment (MCI) or Alzheimer's disease (AD).

11-20. (canceled)
